(19) United States
(12) Patent Application Publication
(10) Pub. No.: US 2006/0030023 A1
(43) Pub. Date: Feb. 9, 2006

(54) Bio-waste enzyme packet

(76) Inventors: Heather K. Somers, Costa Mesa, CA (US); James R. Lusk, Costa Mesa, CA (US)

Correspondence Address: Heather K. Somers, 313 Robin Hood Lane, Costa Mesa, CA, 92627, US

(21) Appl. No.: 10/910,196

(22) Filed: Aug. 3, 2004

(51) Int. Cl. C12N 1/20

(52) U.S. Cl. 435/252.5; 435/262.5

(57) ABSTRACT

The present invention discloses a disposable kit and methods for treating bio-waste in a bio-waste containing article, comprising a batch of fluid activatable enzyme-producing bacteria with an amount of the enzyme produced biologically effective to degenerate the bio-waste, a biodegradable carrier that is loaded with the batch of the bacteria, and an encasement that encloses the carrier and the loaded batch of the bacteria.

### FIELD OF THE INVENTION

The present invention relates to kits and methods for treating bio-waste containing articles. More particularly, the invention relates to a disposable kit containing fluid activatable enzyme-producing bacteria to effectively degenerate the bio-waste in bio-waste containing articles.

### BACKGROUND OF THE INVENTION

The bio-waste containing articles, such as soiled diapers, adult incontinence briefs and the like are generally disposed of without any pretreatment. The bio-waste in the bio-waste containing articles are a nuisance that may create health problems during storage, transportation and disposal at a landfill. The problem becomes more severe in a hot summer. Therefore, it is beneficial to treat the bio-waste in bio-waste containing articles at an appropriate time to lessen the environmental problem.

U.S. Pat. No. 6,639,119, entire contents of which are incorporated herein by reference, discloses an absorbent article adapted to receive feces having an effective concentration of a feces modifying agent disposed in the article such that the feces modifying agent is available to contact the feces deposited in the article and to physically or chemically modify the feces deposited in the article wherein the feces modifying agent is selected from the group consisting of: sulfites, thiols, alcohols, mercaptoacetic acid, sodium thioglycolate, thiolactic acid, thioglycoamide, glycerol monothioglycolate, borohydrides, tertiary amines, thiocyanates, thiosulfates, cyanides, thiophosphates, arsenites, phosphines, betaines, and hydroxylamine.

U.S. Pat. No. 6,573,085, entire contents of which are incorporated herein by reference, discloses a waste bio-argumentation system that adjusts the environment to be treated to a condition that is more conducive for bio-degradation of waste by introducing activated microorganisms designed for that purpose.

U.S. Pat. No. 6,325,934, entire contents of which are incorporated herein by reference, discloses sewage waste digestive bacteria and enzymes incorporated into a slow release material and delivered to the site of the waste to digest the solid waste.

U.S. Pat. No. 5,998,695, entire contents of which are incorporated herein by reference, discloses an absorbent article adapted to receive feces having an effective concentration of an ionic complexing feces modifying agent disposed in the article such that the ionic complexing feces modifying agent is available to change the viscosity or hardness of the feces, wherein the ionic complexing feces modifying agent is selected from the group consisting of ZnO, MgO, MnO, CaO, calcium hydroxide, ethanolamines, Al₂O₃, alginates, zinc salts, aluminum salts and combinations thereof.

U.S. Pat. No. 5,719,115, entire contents of which are incorporated herein by reference, discloses enzyme granules comprising enzyme, an inorganic or organic carrier material and a uniform outer pigment-containing coating layer on the granules, the outer coating layer consisting inorganic pigment, an alcohol or alcohol mixture, an emulsifier for the alcohol or alcohol mixture, a dispersant for the pigment and water.

U.S. Pat. No. 5,637,219, entire contents of which are incorporated herein by reference, discloses a multi-stage process for the digestion or conversion of organic matter including supplying organic material-containing liquid feed stock mixture and causing micro organic or catalytic agents to digest or otherwise convert the organic matter to form liquid and/or gaseous by-product(s).

U.S. Pat. No. 5,589,164, entire contents of which are incorporated herein by reference, discloses biowaste treatment agents for treating biowastes in a manner which keeps noxious and toxic substances from being released from the biowaste, and neutralizes such substances released during the course of stabilizing the biowaste. The treatment agents include a surfactant, a metal component, the metal component including a source of zinc, or copper, or a combination of copper with aluminum or iron, and an aldehyde.

U.S. Pat. No. 5,543,309, entire contents of which are incorporated herein by reference, discloses a carrier that contains bacteria and/or enzymes for degrading sewage sludge. The carrier can be in the form of a gel containing coloring matter and optionally a deodorant, or in the form of a core for a roll of toilet tissue or a roll of towels. Enzymes and/or bacteria can be in a slurry of cellulose pulp used to make the core, in the bonding agent, or in a coating or strip on an inside and/or outside layer. The tube is readily breakable into a plurality of small pieces that can be flushed down a toilet bowl into a sewage system where the pieces disintegrate and release the enzymes and/or bacteria.

U.S. Pat. No. 5,275,943, entire contents of which are incorporated herein by reference, discloses tablets that release components over time for biological degradation of organic matter such as sewage sludge, petroleum hydrocarbons, pesticides and herbicides. The tablets contain an inner-core of a dormant live microorganism, an inner-coating over the inner-core of water soluble hydroxypropyl methylcellulose or polyethylene glycol, an outer-layer over the inner-coating of sodium sulfate coated sodium carbonate peroxyhydrate particles, and an outer-coating over the outer-layer of water soluble hydroxypropyl methylcellulose or polyethylene glycol. The outer-layer may contain additives such as enzymes, buffering agents, sugars and manganese dioxide as an oxidation catalyst. When the tablets are placed in an aqueous environment, layers of the tablets dissolve over time releasing components therein.

U.S. Pat. No. 5,100,800, entire contents of which are incorporated herein by reference, discloses a process for biologically treating chemical process wastewater and chemical waste landfill leachate or for the microbial degradation of toxic halogenated organic chemical waste, which comprises applying an aqueous medium containing a microorganism selected from the group consisting of Pseudomonas putida strain UNK-1 having accession number ATCC 68832 mutants thereof, where the microorganism is capable of degrading the compounds in the aqueous medium.

U.S. Pat. No. 5,089,407, entire contents of which are incorporated herein by reference, discloses biological materials such as enzymes, proteins and peptides being encapsulated by forming a mixture of the material and an aqueous non-ionic polymer solution, spraying the mixture into a circulating water-immiscible nonsolvent for the polymer at a temperature sufficient to freeze the beads and drying the frozen beads to remove essentially all unbound water. Suitable non-ionic polymers are poly(vinyl alcohol), polyvinylpyrollidone, dextran and derivatized cellulose.

U.S. Pat. No. 4,810,385, entire contents of which are incorporated herein by reference, discloses a device suitable for seeding bacterial cultures to waste flowing through or which has accumulated in a collection system which comprises a porous outer covering member which forms an enclosed package with a source of bacterial cultures contained within the package. The cultures suitable for seeding a collection system as a waste stream flows through the porous covering member of the enclosed package causes the bacteria to be released into the waste stream.

The prior art teaches certain bio-waste treatment agent or enzyme-producing bacteria (E-PB) with enzymes capable of degrading the target bio-waste. None of them discloses a disposal kit and methods for treating bio-waste in a bio-waste containing article by providing and placing a disposable treatment kit that comprises a batch of fluid activatable enzyme-producing bacteria to effectively degenerate the bio-waste at a desired location of a pre-soiled article. What is needed is a treatment kit that is relatively inexpensive and readily available, which can be applied by the user to manage the bio-waste problems derived from bio-waste containing articles.

### SUMMARY OF THE INVENTION

It is one object of the present invention to provide a disposable kit for treating bio-waste in a bio-waste containing article, comprising: a batch of fluid activatable enzyme-producing bacteria, wherein the amount of the enzyme produced is biologically effective to degenerate the bio-waste; and a biodegradable carrier, wherein the carrier is loaded with the batch of the bacteria. In a further embodiment, the kit comprises an encasement that encloses the carrier and the loaded batch of the bacteria.

In some aspects of the invention, the encasement of the disposable kit is made of fluid impervious material sized and configured to enclose the carrier and the loaded batch of the bacteria. In a further embodiment, the encasement is a sealed plastic encasement or a free standing container made of metal or plastic. In one alternate embodiment, the disposable kit further comprises an adhesive strip applied to at least one outer edge of the encasement, the adhesive strip being a non-adhesive pull-tab for easy removal of the disposable kit containing the carrier and the loaded batch of the bacteria, wherein the adhesive strength of the adhesive strip onto the encasement is preferably between 0.5 psi and 1.5 psi. In a preferred embodiment, an outer edge of the encasement is heat-sealed, the heat-sealed edge having an exposed pull-apart tab for easy removal of the disposable kit. The encasement may be selected from a group consisting of a blister-pack, a plastic pack, a foil-type paper-backed pack, and a vacuum sealed pack. The encasement may be made of a hermetically sealed material selected from a group consisting of plastic, foil, wax faced paper, and mylar-type material that may be torn or cut to be opened.

In some further embodiments, the carrier comprises gelatin, cellulose, woven cellulose, or meshed cellulose that is capable of encapsulating between about 0.1 gram and 5.0 grams dry weight of E-PB. In still another embodiment, the carrier comprises a gel-cap that contains E-PB in a semi-fluid or gelatinous form.

Some aspects of the present invention relate to a bio-waste degrading, enzyme-producing bacteria similar in form and function to Zep Commercial Septic System and Cesspool Treatment to be henceforth referred to as E-PB. In one embodiment, the enzyme-producing bacteria comprises genus Bacillus. In another embodiment, the enzyme-producing bacteria comprises genus Bacillus having the ability to produce polyphenol oxidase. In still another embodiment, the enzyme-producing bacteria is selected from a group consisting of Bacillus alcalophilus, Bacillus amyloliquefaciens, Bacillus brevis, Bacillus firmus, Bacillus licheniformis, Bacillus natto, Bacillus pumilus, Bacillus sphaericus, and Bacillus subtilis. In a further embodiment, the enzyme-producing bacteria is selected from a group consisting of Bacillus pumilus ATCC 202136, Bacillus subtilis ATCC 202138, Bacillus amyloliquefaciens ATCC 202134, Bacillus macerans ATCC 202132, Bacillus subtilis ATCC 202137, Bacillus subtilis ATCC 202139, Bacillus amyloliquefaciens ATCC 202133, and Bacillus macerans ATCC 202135.

Some aspects of the invention relate to a disposable kit for treating bio-waste in a bio-waste containing article, wherein the bio-waste containing article is selected from a group consisting of a diaper, a protective underpad, an adult incontinence article, a protective substrate, a colostomy bag, a pet defecation container, a back-packing hygiene gear, a military field operations hygiene pack, a feminine hygiene article, and the like.

In further embodiments, the disposable kit for treating bio-waste in a bio-waste containing article is in a form of capsule, tablet, gel-cap, powder, and combination thereof, wherein the tablet is manufactured by a method of freeze-drying processes, and wherein the tablet contains between about 0.1 gram and 5.0 grams dry weight of E-PB.

Some aspects of the invention relate to a dispensing box for dispensing at least one disposable bio-waste treatment kit, comprising a container sized and configured for stacking of the at least one disposable kit, wherein the kit comprises a batch of fluid activatable enzyme-producing bacteria, a biodegradable carrier, wherein the carrier is loaded with the batch of the bacteria, and an encasement that encloses the carrier and the loaded batch of the bacteria.

In some embodiments, the dispensing box is a wall-mounted dispenser, a free standing dispenser, or a convertible dispenser between the wall-mounted and free standing fashions. In a further embodiment, any of the dispensers has a thumb notch cut out at the bottom of the box configured for dispensing the at least one disposable bio-waste treatment kit. In a further embodiment, any of the dispensers has a hinged lid at the top of the box configured for enclosing the at least one disposable bio-waste treatment kit inside the box. In a further embodiment, any of the dispensers has a notched hole at the rear portion of the box configured for sliding the box over a prior wall-mounted screw or nail, wherein the wall-mounted screw or nail is L-shaped with the L-arm pointing upward. In a further embodiment, any of the dispensers is made of a pre-formed durable plastic or metal with a dimension of between about ⅛ inch and 1/16 inch thickness.

Some aspects of the invention relate to a method of treating bio-waste in a bio-waste containing article, comprising: providing a disposable treatment kit that comprises a batch of fluid activatable enzyme-producing bacteria, wherein the amount of the enzyme produced is biologically effective to degenerate the bio-waste, and a biodegradable carrier loaded with the batch of the bacteria; placing the kit at a desired location of a pre-soiled article, wherein the pre-soiled article is the bio-waste containing article before being loaded with the bio-waste; and treating the bio-waste by releasing the fluid activated enzyme from the bacteria. In a further embodiment, the pre-soiled article further comprises at least one absorbent layer, the desired location being at the at least one absorbent layer. In a further embodiment, the carrier is a biodegradable cellulose film or a time-release woven cellulose pack and the disposable treatment kit further comprises an encasement that encloses the carrier and the loaded batch of the bacteria, wherein the encasement is adhesively or mechanically attached to the bio-waste containing article.

### BRIEF DESCRIPTION OF THE DRAWINGS

Further features and advantages of the present invention will become apparent to one of skill in the art in view of the Detailed Description of Exemplary Embodiments that follows, when considered together with the attached drawings and claims.

FIG. 1A shows an exterior perspective view of a diaper article.

FIG. 1B shows a side view of the diaper article of FIG. 1.

FIG. 2A shows an individually wrapped kit containing the E-PB formed substrate.

FIG. 2B shows a side view of the individually wrapped kit of FIG. 2A.

FIG. 2C shows an individually wrapped kit containing the E-PB loose powder.

FIG. 2D shows a side view of the individually wrapped kit of FIG. 2C.

FIG. 3A shows an individually wrapped E-PB containing kit embedded inside a diaper article.

FIG. 3B shows a side view of the individually wrapped E-PB containing kit embedded inside a diaper article of FIG. 3A.

FIG. 4A shows the E-PB embedded inside a diaper article in a loose powder form.

FIG. 4B shows a side view of the E-PB embedded inside the diaper article of FIG. 4A.

FIG. 5 shows the E-PB in a blister pack container.

FIG. 6A shows a box dispenser for individually wrapped E-PB kits.

FIG. 6B shows one embodiment of a rear view of the box dispenser of FIG. 6A.

FIG. 7A shows another embodiment of a rear view of the box dispenser of FIG. 6A.

FIG. 7B shows an attachment element for hanging the box dispenser of FIG. 7A.

FIG. 8A shows one embodiment of a bottle container for dispensing liquid E-PB.

FIG. 8B shows one embodiment of a spray bottle for dispensing liquid E-PB.

FIG. 8C shows one embodiment of a pump spray bottle for dispensing liquid E-PB.

### DETAILED DESCRIPTION OF EXEMPLARY EMBODIMENTS

The preferred embodiments of the present invention described below relate particularly to a disposable kit for treating bio-waste comprising a batch of fluid activatable enzyme-producing bacteria to effectively degenerate bio-waste in an article. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

As used herein, the term “bio-waste containing article” refers to devices which absorb and contain body exudates, and more particularly, refers to devices which are placed against or in proximity to the body of the wearer to absorb and contain the various exudates discharged from the body. The term “pre-soiled article” refers to the bio-waste containing article before being contaminated with the bio-waste. In general, the bio-waste containing article of the present invention includes, but not limited to, a diaper, a protective underpad, an adult incontinence article, a protective substrate, a colostomy bag, a pet defecation container, a back-packing hygiene gear, a military field operations hygiene pack, a feminine hygiene article, and the like. The term “E-PB” is herein intended to mean the enzyme-producing bacteria.

It is the intent of this invention to substantially reduce the amount of untreated bio-waste contained in disposable articles using naturally occurring enzyme-producing bacteria encapsulated in a fluid soluble capsule or tablet, spray mist, aerosol, powdered form, or the like. In alternative embodiments, the enzyme-producing bacteria could be used with enzymatic surfactants to further enhance the process of waste reduction or treatment in uses such as kitchen trash bags or bio-hazard waste disposal systems.

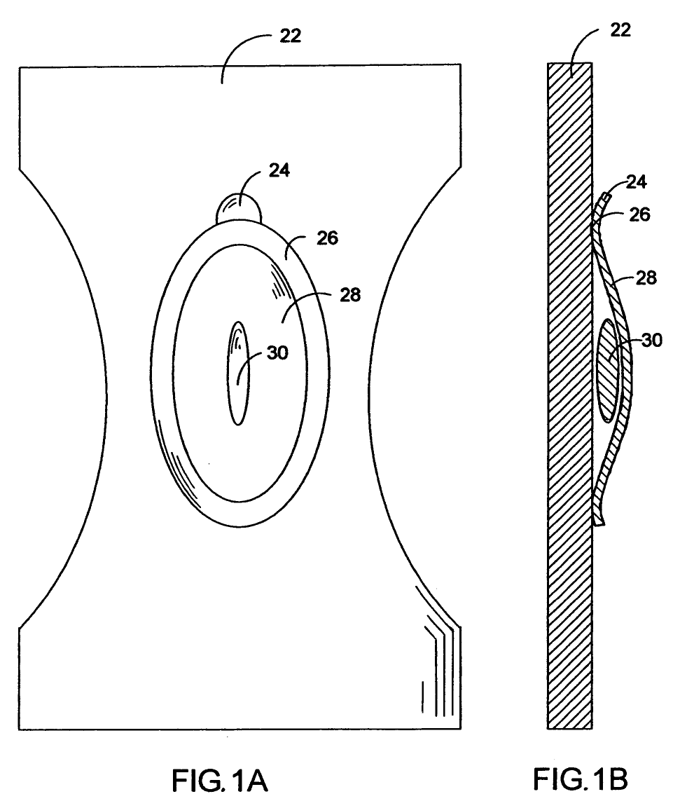

In one embodiment, the enzyme-producing bacteria (E-PB) may be contained in a fluid-soluble, bio-degrading gel-cap capsule that contains the E-PB in a fluid, a semi-fluid or gelatinous form. FIG. 1A shows an exterior perspective view of a diaper article with one layer 22 or a pre-soiled article whereas FIG. 1B shows a side view of the article of FIG. 1A. A disposable E-PB kit 30 is placed at about the middle of the layer 22 enclosed under fluid impervious containment material 28 that can be any shape conducive to containment and dispensing of the enzyme content. In one preferred embodiment, the E-PB kit is placed at either the exterior or interior surface of the diaper article. In one embodiment, the E-PB kit 30 is under the containment material that has an adhesive edge 26 to seal the containment material and a non-adhesive pull tab 24. The E-PB kit may be in a form of capsule, tablet or other appropriate solid form having the E-PB loaded with a carrier. In another embodiment, the E-PB kit is preferably placed at an exterior side of the diaper article. In some embodiment, the carrier is an inactive ingredient. In another embodiment, the carriers comprises at least one auxiliary ingredient, such as surfactants, builders, and the like to enhance the effectiveness of biodegrading the target bio-waste. Some aspects of the invention relate to a disposable kit for treating bio-waste, comprising: a batch of fluid activatable enzyme-producing bacteria, wherein an amount of the enzyme produced is biologically effective to degenerate the bio-waste; and a biodegradable carrier containing the batch, wherein the carrier is placed at a bio-waste containing article for treating the bio-waste. In one embodiment, the carrier comprises gelatin, cellulose, and the like.

In one preferred embodiment, the enzyme-producing bacteria is in a form of a freeze-dried tablet with up to ten grams or more dry weight E-PB, preferably between about 0.1 gram and about 5.0 grams dry weight of E-PB bacteria. In another embodiment, the enzyme-producing bacteria is a fluid soluble, biodegradable woven cellulose bag or other biodegradable encasement capable of containing up to ten grams or more of dry weight powdered E-PB, preferably between about 0.1 gram and about 5.0 grams dry weight of E-PB bacteria. In a further embodiment, the enzyme-producing bacteria is in a fluid activatable, loose powdered form sufficient enough in quantity of up to ten grams or more dry weight, preferably between about 0.1 gram and about 5.0 grams dry weight of E-PB bacteria to degenerate the target bio-waste contained in disposable articles.

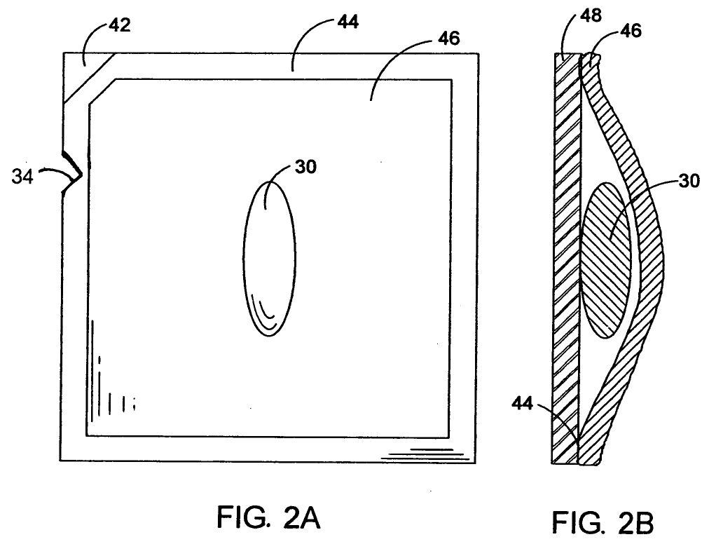

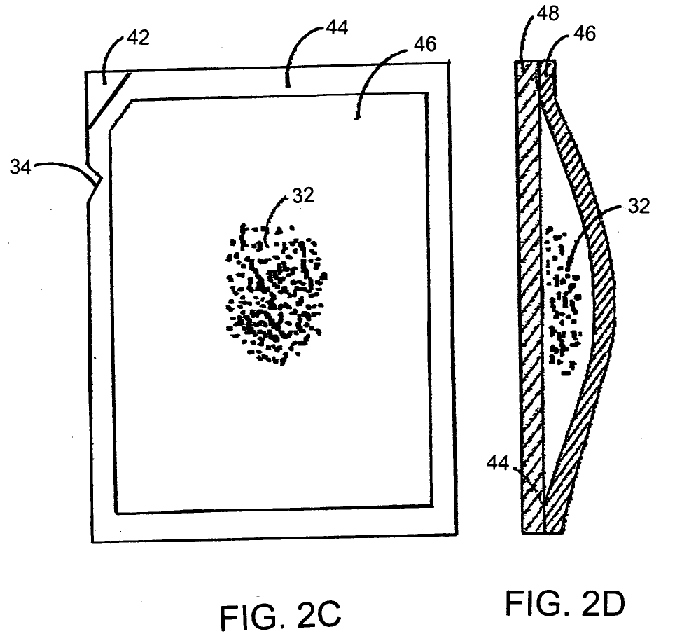

FIG. 2A shows an individually wrapped kit containing the E-PB formed substrate whereas FIG. 2B shows a side view of the individually wrapped kit of FIG. 2A. Further, FIG. 2C shows an individually wrapped kit containing the E-PB loose powder whereas FIG. 2D shows a side view of the individually wrapped kit of FIG. 2C. The formed substrate herein includes tablet, capsule, pellet, gel-cap, and the like. By way of illustration, the enzymes from the E-PB react with the bio-waste (i.e. feces, blood, urine) to enzymatically break down the material and reduce the levels of untreated bio-waste in landfills, effluents, tributaries, and oceans. The broken-down byproducts following the bio-waste treatment of the present invention are generally more environmentally acceptable.

When the encapsulated E-PB container or packet is exposed to the water in bio-waste, in particular, fecal or hemoglobin-based material, the fluid dissolves the gelatin or cellulose container exposing the enzyme-producing bacteria to the bio-waste. The bacteria feeds on the bio-waste producing an enzyme or enzymes which degrades at least a portion of the bio-waste. The bio-waste decomposes into a more eco-friendly state between the time it is exposed to the E-PB and the time it is placed in the landfill or other disposal means. Even while in the landfill, the enzymes continue to decompose the bio-waste, further reducing the impact on the environment. Exposure of the E-PB to any bio-waste triggers an enzymatic reaction from the bacteria thus inducing a reduction of the bio-waste to more basic forms of carbon, nitrogen and minerals, and more eco-friendly, re-absorbable waste by-products.

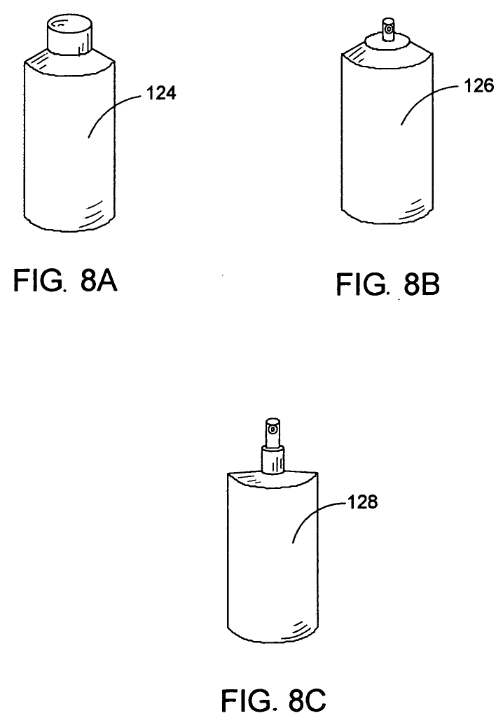

The E-PB may be contained or loaded in many forms and packaged for distribution and application in any manner. For example, a fluid impervious sealed plastic or other fluid impervious material for outer encasement is sized and configured to contain the encapsulated E-PB material with a dimension of approximately 3×3 inches. The encasement with the E-PB in any form of capsule 30, tablet, powdered 32 or other container may be adhesively attached to outside of the pre-soiled article selected from a group consisting of a diaper, a protective underpad, an adult incontinence article, a protective substrate, a colostomy bag, a pet defecation container, a back-packing hygiene gear, a military field operations hygiene pack, a feminine hygiene article, and the like. The encasement may be a sealed plastic encasement or a free standing container made of metal or plastic. The E-PB may also be in the form of a fluid that could be dispensed through drops, an atomizer, plastic or metal bottle 124, spray bottle 126, pump spray bottle 128, or aerosol container as shown in FIGS. 8A-8C. The E-PB may be contained in either dry, powdered, or within a fluid, semi-fluid or gelatinous consistency contained within a gelatin or other fluid pervious or fluid degrading vessel or spray.

For the E-PB that is encased in a fluid impervious container 28 attached to the exterior of a substrate or layer 22 as shown in FIG. 1A and FIG. 1B, an adhesive or heat-sealing 26 is applied to the outer one quarter (¼) to one eighth (⅛) inch of the edge of the fluid impervious encasement. In one embodiment, the adhesive or heat-sealing 44 is applied to the outer encasement 46 as shown in FIGS. 2A-2D. In another embodiment, the fluid impervious encasement may also be heat sealed onto a substrate with a non-sealed, exposed pull-tab for easy removal of the content. The adhesive strength of the adhesive strip onto the encasement is between 0.3 psi and 2.5 psi, preferably between about 0.5 psi and 1.5 psi. In one alternate embodiment, individual packaging or kits of the invention may also include, but not be limited to, two fluid-impervious (possibly plastic) type enclosure sheets bound together by approximately ⅛-¼ inches of adhesive around the entire exterior edges of the containment enclosure. In some embodiments as shown in FIGS. 1B, 2B, 2D, 3B, and 4B, the diaper article or other absorbent article may have a plurality of layers whereby at least one liquid pervious layer is at the outer-most exterior side. In a further embodiment, the inner layer(s) may be liquid impervious.

Individualized packaging or kits consisting of a fluid impervious encasement sealed by either method of adhesion or heat sealed may be opened by methods of tearing apart, cutting, or pulling apart the sealed edges. In one aspect of the invention, it is provided a tear or cut notch 34 on the encasement 44. In another aspect, it is provided a corner non-adhesive flap 42 on the encasement for pulling apart the sealed encasement.

Other forms of individualized packaging or kits may consist of a blister-pack, plastic and/or foil-type, paper-backed, vacuum sealed, fluid-impervious container to ease handling and/or dispensing E-PB in any type of cellulose mesh container form. FIG. 5 illustrates one example of several blister pack containers 122 in one packaging, wherein each container holds the E-PB in its desired form.

Individualized packets or kits may consist of a vacuum sealed pull-apart fluid impervious plastic-type material adhesively or heat sealed with pull apart tabs.

Individualized packets or kits may consist of hermetically sealed, fluid-impervious materials such as plastic, foil, wax faced paper, and mylar-type material that may be torn or cut to be opened at designated notch or pulling-apart corner flap. A non-adhesive pull-tab would allow for ease of separation of adhesive of fluid-impervious containment enclosure away from substrate or to separate two adhesive sides of the fluid impervious enclosure in instances of individually wrapped E-PB kits.

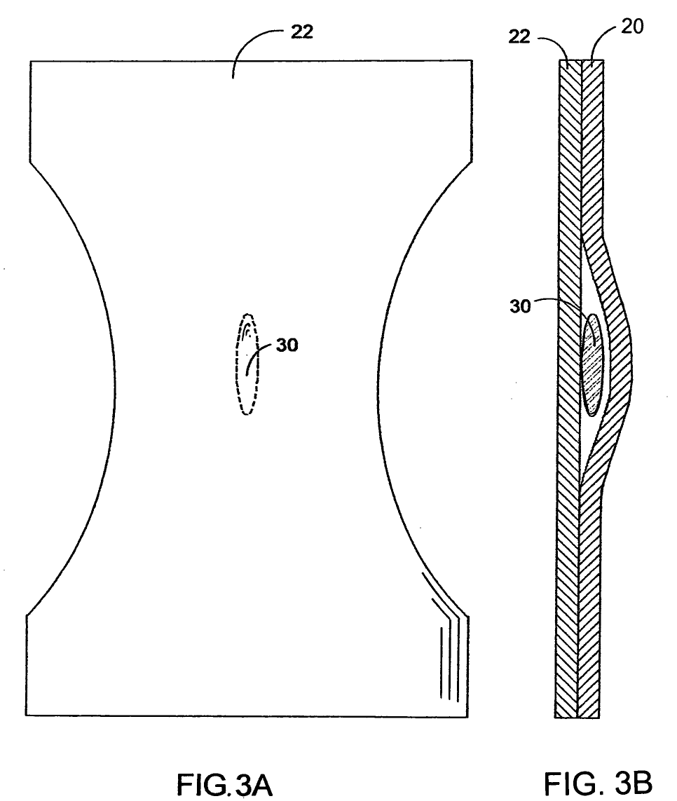

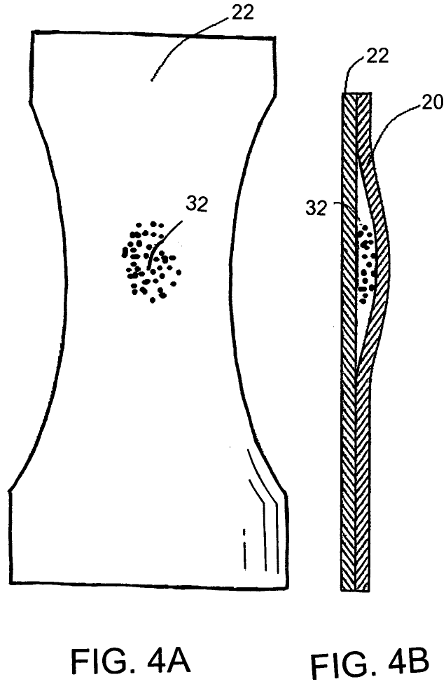

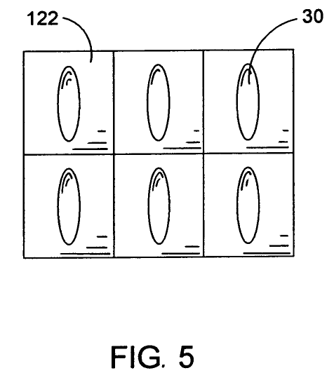

FIG. 3A shows an individually wrapped E-PB containing kit embedded inside a diaper article whereas FIG. 3B shows a side view of the individually wrapped E-PB containing kit embedded inside a diaper article of FIG. 3A. The diaper is consisted of an inner layer 20 that is secured to an outer layer 22. In a further embodiment, FIG. 4A shows the E-PB embedded inside a diaper article in a loose powder form whereas FIG. 4B shows a side view of the E-PB imbedded inside the diaper article in a loose powder form of FIG. 4A. In one embodiment, the E-PB is encased in the absorbent layer of the substrate (i.e. disposable diaper or underpad, etc.) enclosed in a layer of semi-pervious time release, fluid activated woven or bio-degradable cellulose film. In some embodiments, the exterior layer 20, 46 is preferably pervious while the interior layer 22, 48 is impervious.

The E-PB may also be contained in a bottle, jar, vial, can, or other liquid dispenser and used as a wet spray, aerosol, drops or atomized. The E-PB is safely and commonly used today in septic tanks, cesspools and portable waste disposal units. The E-PB may also digest paper and helps to stop unpleasant odors.

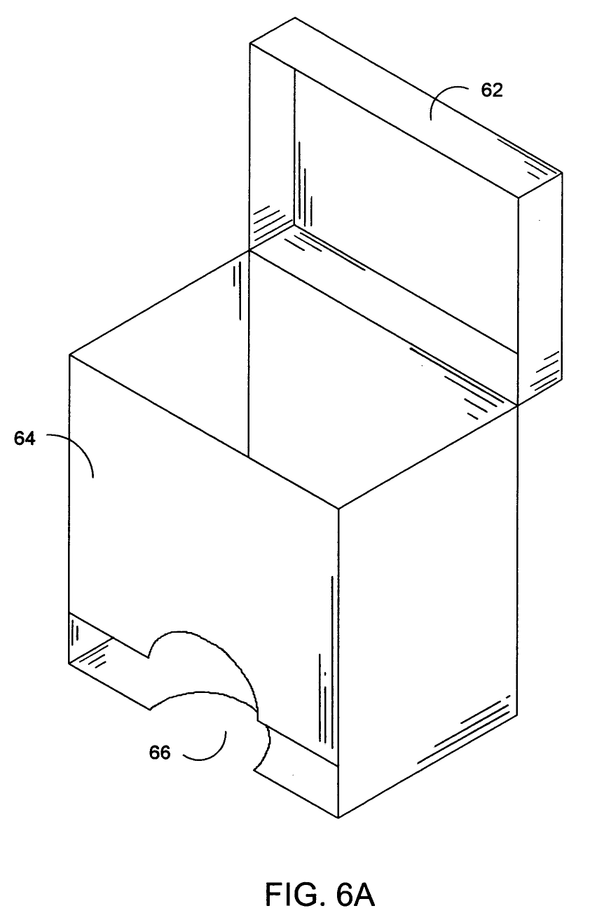

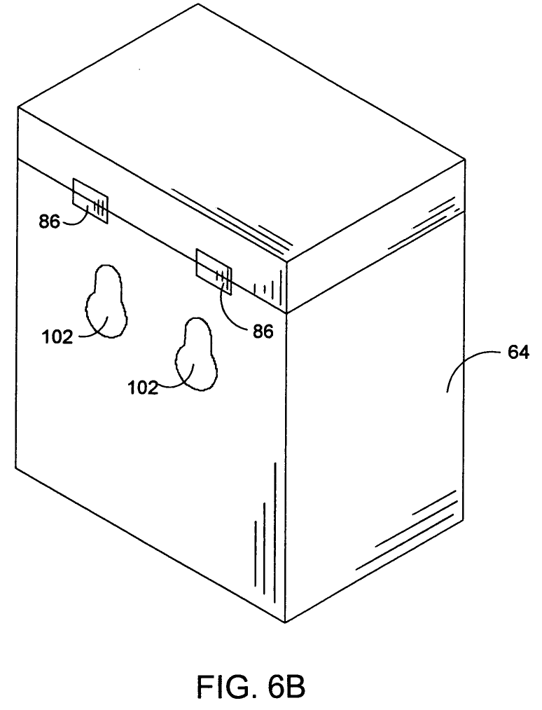

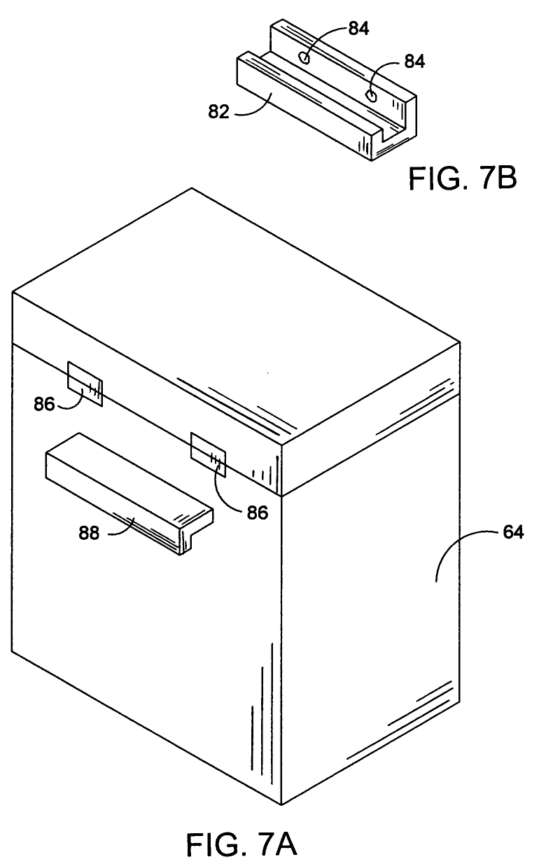

FIG. 6A shows a box dispenser 64 for individually wrapped E-PB kits whereas FIG. 6B shows one embodiment of a rear view of the box dispenser of FIG. 6A. FIG. 7A shows another embodiment of a rear view of the box dispenser of FIG. 6A whereas FIG. 7B shows an attachment element for hanging the box dispenser of FIG. 7A. Some aspects of the present invention relate to a dispensing box for dispensing at least one disposable bio-waste treatment kit, comprising a container sized and configured for stacking of the at least one disposable kit, wherein the kit comprises a batch of fluid activatable enzyme-producing bacteria, a biodegradable carrier, wherein the carrier is loaded with the batch of the bacteria, and an encasement that encloses the carrier and the loaded batch of the bacteria.

In a further embodiment, the dispensing box is made of a pre-formed durable plastic or metal with a dimension of between about ⅛ inch and 1/16 inch thickness. Other thickness is also applicable. In one embodiment, the dispensing box is a wall-mounted dispenser (FIG. 6B), a free standing dispenser (FIG. 6A), or a convertible dispenser between the wall-mounted and free standing fashions (FIG. 7A). In another embodiment, the dispenser or dispensing box has a thumb notch 66 cut out at the bottom of the box 64 configured for dispensing the at least one disposable bio-waste treatment kit. In a further embodiment, the dispenser has a hinged lid 62 with a couple of hinges 86 configured for enclosing the at least one disposable bio-waste treatment kit inside the box. The content of the kit is selected from a group consisting of capsule, tablet, gel-cap, container, and combination thereof. In another embodiment, the form of the content of the kit is powder.

In some embodiment, the dispensing box has a slotted hole 102 at the rear portion of the box 64 configured for sliding the box over a prior wall-mounted screw or nail, wherein the wall-mounted screw or nail is L-shaped with the L-arm pointing upward. In an alternate embodiment, the dispensing box has a wall attachment 88 at a rear portion of the box 64 configured for sliding the box over or lifting off a prior wall-mounted matching slide-wall attachment 82 that is securely mounted to the wall though nail holes 84.

Enzymes

Some aspects of the present invention relate to a bio-waste degrading, enzyme-producing bacteria (referred to herein as E-PB) loaded or enclosed in a carrier for treating bio-waste. In one embodiment, the E-P B is similar in form and function to Zep Commercial Septic System and Cesspool Treatment (manufactured by Zep Manufacturing Company, Atlanta, Ga.). The enzyme-producing bacteria of the present invention is described in more details below.

Enzymes are biological catalysts in the form of globular proteins that drive chemical reactions in the cells of living organisms. As such, they are involved in the metabolic demands of an extensive range of cell types, whereby the enzyme reactions take place under mild conditions, specific according to enzyme class with very fast reaction rates. Among the applications, bio-waste degradation with an appropriate enzyme is environmentally beneficial. An enzyme usually catalyzes only one specific chemical reaction or a number of closely related reactions. Unlike non-enzymatic chemical reactions, enzyme reactions rarely lead to the formation of waste by-products.

Enzymes accelerate reactions generally by factors of at least a million. For example, carbonic anhydrate, which catalyzes the hydration of carbon dioxide to speed up its transfer in aqueous environments like the blood, is one of the fastest enzymes known. Each molecule of the enzyme can hydrate 100,000 molecules of carbon dioxide per second. This is equivalent to ten million times faster than a non-enzyme-catalyzed reaction.

Most enzymes catalyze the transfer of electrons, atoms or functional groups. As depending on the types of reaction catalyzed, they are divided into six main classes, which in turn are split into groups and subclasses. The six main classes of enzyme are oxidoreductases, transferases, hydrolases, lyases, isomerases, and ligases, of which hydrolases are the main enzymes that is biologically effective to degenerate the bio-waste of the present invention.

Like other proteins, enzymes are produced inside cells by ribosomes, which link up amino acids into chains. During and after their formation by ribosomes, the primary chains of amino acid residues undergo a controlled folding process to form a three-dimensional protein that has a major bearing on the finished enzyme's catalytic specificity and activity. The majority of industrial enzymes are produced by microorganisms (for example, bacteria). Enzymes has molecular weights ranging from about 12,000 to over 1 million Daltons and demand physical space for movement and to be able to act on the much smaller functional groups in substrate.

Most industrial enzymes are produced using microorganisms. The enzyme-producing organisms belong to either the genus Bacillus (gram-positive bacteria) or to the genus Aspergillus (filamentous fungi).

By ways of illustration, enzymes are used as functional ingredients in detergents that clean laundry and dishes. They work in an efficient, environmentally sound and energy-saving way. Similarly, enzymes can be used to degrade bio-waste in bio-waste containing article, such as an absorbent diaper. The most widely used detergent enzymes are hydrolases, which remove soils formed from proteins, lipids and polysaccharides. Cellulase is a type of hydrolase that provides fabric care through selective reactions not previously possible when washing clothes. Some aspects of the invention relate to a disposable kit for treating bio-waste, comprising: a batch of fluid activatable enzyme-producing bacteria, wherein an amount of the enzyme produced is biologically effective to degenerate at least a portion of the bio-waste; and a biodegradable carrier containing the batch of E-PB, wherein the carrier is placed within or at a bio-waste containing article for treating the bio-waste.

U.S. Pat. No. 4,945,053, entire contents of which are incorporated herein by reference, discloses alkaline cellulase obtained by isolation from a culture product of Bacillus sp. These enzymes are active in an alkaline as well as in neutral or acidic laundry detergent solution. Finding of an alkaline cellulase derived from alkaliphilic microorganisms belonging to Bacillus sp. enabled its use for laundry heavy-duty detergents and treating the bio-waste. Certain enzyme-producing bacteria could be used as a disposable kit for treating bio-waste of the present invention.

Recent progress in genetic engineering has enabled mass production of enzymes for detergents, which also applies to the production of alkaline cellulases. A number of genes for alkaline cellulases have already been cloned, their nucleotide sequences have been determined. Furthermore, technique for mutagenesis and breeding of the enzyme-producing bacteria or mutagenesis of a gene encoding the enzyme has been introduced. In some aspects of the present invention, various enzymes in addition to the cellulase may be incorporated. Examples of the enzyme include hydrolases, oxidases, reductases, transferases, lyases, isomerases, ligases and synthetases. Of these, cellulases, proteases, keratinases, esterases, cutinases, amylases, lipases, pullulanases, pectinases, mannases, glucosidases, glucanases, cholesterol oxidases, peroxidases, and laccases are preferred. Among them, proteases, cellulases, amylases and lipases are especially preferred.

Enzyme-Producing Bacteria

Enzyme-producing bacteria or microbes require an energy producing system (including an electron acceptor) to sustain life and nutrients, including liquid water, in order to grow and reproduce. Since microbes have been present on earth longer than other organisms, they have evolved the ability to thrive in almost any environment that meets these minimal criteria. Energy comes from one of two sources, light (photosynthesis) or the oxidation of reduced molecules. Oxidizable molecules may be organic (e.g. sugar, protein or any of the other foods we humans relish) or a variety of inorganic molecules such as sulfur, iron, hydrogen, carbon monoxide, or ammonia or even a combination of organic/inorganic molecules.

U.S. Pat. No. 6,184,014, entire contents of which are incorporated herein by reference, discloses some of the enzyme-producing bacteria. According to the cited invention, polyphenol oxidase is derived from a bacterial strain, preferably a strain of the genus Bacillus. Any and every strain of the genus Bacillus having the ability to produce polyphenol oxidase can be used to obtain the polyphenol oxidase. The enzyme-producing bacteria employable may include, for example, those of Bacillus alcalophilus, Bacillus amyloliquefaciens, Bacillus brevis, Bacillus firmus, Bacillus licheniformis, Bacillus natto, Bacillus pumilus, Bacillus sphaericus, and Bacillus subtilis, etc. Certain enzyme-producing bacteria could be used as a disposable kit for treating bio-waste of the present invention.

U.S. Pat. No. 6,177,012, entire contents of which are incorporated herein by reference, discloses some of the enzyme-producing bacteria that have been deposited with the American Type Culture Collection (“ATCC”), 10801 University Blvd., Manassas, Va., 20110-2209, U.S.A., on Jun. 5, 1998, under the rules of the Budapest Treaty and are designated as follows: Bacillus pumilus ATCC 202136, Bacillus subtilis ATCC 202138, Bacillus amyloliquefaciens ATCC 202134, Bacillus macerans ATCC 202132, Bacillus subtilis ATCC 202137, Bacillus subtilis ATCC 202139, Bacillus amyloliquefaciens ATCC 202133, and Bacillus macerans ATCC 202135. Certain enzyme-producing bacteria could be used as a disposable kit for treating bio-waste of the present invention.

Further, The eight novel Bacillus strains shown above have a variety of potential uses, including treatment of wastewater, such as municipal, food processing, and residential wastewater; pretreatment of wastewater that might contain high levels of oleaginous matter; treatment of on site sewage disposal systems, such as septic tanks and cesspools; agricultural uses, including livestock manure waste management or as Direct Fed Microbials for use in livestock feeds to increase food digestion and utilization; and for treatment of pipes, traps, plumbing systems and grease traps. Some aspects of the present invention provide a method of treating bio-waste in a bio-waste containing article, comprising: providing a disposable treatment kit that comprises a batch of fluid activatable enzyme-producing bacteria, wherein an amount of the enzyme produced is biologically effective to degenerate the bio-waste, and a biodegradable carrier containing the batch; placing the carrier with the batch at a desired location of a pre-soiled article, wherein the pre-soiled article is the bio-waste containing article before being loaded with the bio-waste; and treating the bio-waste by the fluid activated enzyme derived from the bacteria.

From the foregoing description, it should now be appreciated that a disposable kit with a batch of fluid activatable enzyme-producing bacteria that is biologically effective to degenerate the bio-waste in a bio-waste containing article has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention, as described by the appended claims.

What is claimed is:

1. A disposable kit for treating bio-waste in a bio-waste containing article, comprising:
a batch of fluid activatable enzyme-producing bacteria, wherein an amount of the enzyme produced is biologically effective to degenerate said bio-waste; and
a biodegradable carrier, wherein said carrier is loaded with said batch of the bacteria.

2. The disposable kit of claim 1, wherein the carrier comprises gelatin or cellulose.

3. The disposable kit of claim 1, wherein the bio-waste containing article is selected from a group consisting of a diaper, a protective underpad, an adult incontinence article, a protective substrate, a colostomy bag, a pet defecation container, a back-packing hygiene gear, a military field operations hygiene pack, and a feminine hygiene article.

4. The disposable kit of claim 1, wherein the enzyme-producing bacteria comprises genus Bacillus.

5. The disposable kit of claim 1, wherein the enzyme-producing bacteria comprises genus Bacillus having the ability to produce polyphenol oxidase.

6. The disposable kit of claim 1, wherein the enzyme-producing bacteria is selected from a group consisting of Bacillus alcalophilus, Bacillus amyloliquefaciens, Bacillus brevis, Bacillus firmus, Bacillus licheniformis, Bacillus natto, Bacillus pumilus, Bacillus sphaericus, and Bacillus subtilis.

7. The disposable kit of claim 1, wherein the enzyme-producing bacteria is selected from a group consisting of Bacillus pumilus ATCC 202136, Bacillus subtilis ATCC 202138, Bacillus amyloliquefaciens ATCC 202134, Bacillus macerans ATCC 202132, Bacillus subtilis ATCC 202137, Bacillus subtilis ATCC 202139, Bacillus amyloliquefaciens ATCC 202133, and Bacillus macerans ATCC 202135.

8. The disposable kit of claim 1, wherein the kit is in a form of capsule, tablet, gel-cap, and combination thereof.

9. The disposable kit of claim 8, wherein the tablet is manufactured by a method of a freeze-drying process.

10. The disposable kit of claim 8, wherein the tablet contains between 0.1 gram and 5.0 grams dry weight of E-PB.

11. The disposable kit of claim 1, wherein the carrier comprises woven cellulose that is capable of encapsulating between 0.1 gram and 5.0 grams dry weight of E-PB.

12. The disposable kit of claim 1, wherein the carrier comprises a gel-cap that contains E-PB in a fluid, semi-fluid or gelatinous form.

13. The disposable kit of claim 1, wherein the kit is in a powder form.

14. The disposable kit of claim 1, further comprising an encasement that encloses the carrier and the loaded batch of said bacteria.

15. The disposable kit of claim 14, wherein the encasement is made of fluid impervious material sized and configured to enclose the carrier and the loaded batch of said bacteria.

16. The disposable kit of claim 14, wherein the encasement is a sealed plastic encasement or a free standing container made of metal or plastic.

17. The disposable kit of claim 14, further comprising an adhesive strip applied to at least one outer edge of said encasement, the adhesive strip being a non-adhesive pull-tab for easy removal of the carrier and the loaded batch of said bacteria.

18. The disposable kit of claim 17, wherein adhesive strength of said adhesive strip onto said encasement is between 0.5 psi and 1.5 psi.

19. The disposable kit of claim 14, wherein an outer edge of said encasement is heat-sealed, the heat-sealed edge having an exposed pull-apart tab for easy removal of the carrier and the loaded batch of said bacteria.

20. The disposable kit of claim 14, wherein said encasement is selected from a group consisting of a blister-pack, a plastic pack, a foil-type paper-backed pack, and a vacuum sealed pack.

21. The disposable kit of claim 14, wherein said encasement is made of a hermetically sealed material selected from a group consisting of plastic, foil, wax faced paper, and mylar-type material that may be torn or cut to be opened.

22. A dispensing box for dispensing at least one disposable bio-waste treatment kit, comprising a container sized and configured for stacking of the at least one disposable kit, wherein said kit comprises a batch of fluid activatable enzyme-producing bacteria, a biodegradable carrier, wherein said carrier is loaded with said batch of the bacteria, and an encasement that encloses the carrier and the loaded batch of said bacteria.

23. The dispensing box of claim 22, wherein the box is a wall-mounted dispenser, a free standing dispenser, or a convertible dispenser between said wall-mounted and free standing fashions.

24. The dispensing box of claim 23, wherein any of said dispensers has a thumb notch cut out at a bottom of the box configured for dispensing the at least one disposable bio-waste treatment kit.

25. The dispensing box of claim 23, wherein any of said dispensers has a hinged lid at top of the box configured for enclosing the at least one disposable bio-waste treatment kit inside the box.

26. The dispensing box of claim 23, wherein any of said dispensers has a notched hole at a rear portion of the box configured for sliding said box over a prior wall-mounted screw or nail.

27. The dispensing box of claim 26, wherein said wall-mounted screw or nail is L-shaped with the an L-arm pointing upward.

28. The dispensing box of claim 23, wherein any of said dispensers is made of a pre-formed durable plastic or metal with a dimension of between about ⅛ inch and 1/16 inch thickness.

29. A method of treating bio-waste in a bio-waste containing article, comprising:
providing a disposable treatment kit that comprises a batch of fluid activatable enzyme-producing bacteria, wherein an amount of the enzyme produced is biologically effective to degenerate said bio-waste, and a biodegradable carrier loaded with said batch of the bacteria;
placing the kit at a desired location of a pre-soiled article, wherein said pre-soiled article is the bio-waste containing article before being loaded with said bio-waste; and
treating said bio-waste by releasing the fluid activated enzyme from said bacteria.

30. The method of claim 29, wherein the pre-soiled article further comprises at least one absorbent layer, the desired location being at the at least one absorbent layer.

31. The method of claim 29, wherein the carrier is a biodegradable cellulose film or a time-release woven cellulose pack.

32. The method of claim 29, wherein the kit further comprises an encasement that encloses the carrier and the loaded batch of said bacteria.

33. The method of claim 32, wherein said encasement is adhesively attached to the bio-waste containing article.

34. The method of claim 29, wherein the enzyme produced from the enzyme-producing bacteria is timely released to said bio-waste containing article.
